(19) United States
(12) Patent Application Publication
(10) Pub. No.: US 2024/0115415 A1
(43) Pub. Date: Apr. 11, 2024

(54) TONGUE RING AND OTHER TONGUE-ATTACHED DEVICES FOR PREVENTION OF SLEEP APNEA

(71) Applicant: Morteza Naghavi, Long Beach, CA (US)

(72) Inventor: Morteza Naghavi, Long Beach, CA (US)

(21) Appl. No.: 18/483,051

(22) Filed: Oct. 9, 2023

Related U.S. Application Data
(60) Provisional application No. 63/414,557, filed on Oct. 9, 2022.

(51) Int. Cl. A61F 5/56

(52) U.S. Cl. CPC A61F 5/566

(57) ABSTRACT

According to an aspect of the present invention, there is provided a tongue retaining device configured for reducing obstructive sleep apnea, comprising: a tongue piercing device; and one or more threads attached to the tongue piercing device and anchoring it to an anterior anchor point.

### CROSS-REFERENCE

This application claims priority to U.S. Provisional Patent Application 63/414,557, filed Oct. 9, 2022.

### BACKGROUND

Sleep apnea is a serious sleep disorder characterized by repeated pauses in breathing during sleep. These pauses can last anywhere from a few seconds to several minutes and can occur hundreds of times a night.

Sleep apnea can lead to a number of serious health problems, including:

Cardiovascular disease: Sleep apnea is a major risk factor for heart attack, stroke, and heart failure.

Type 2 diabetes: Sleep apnea can make it difficult to control blood sugar levels, increasing the risk of developing type 2 diabetes.

Obesity: Sleep apnea can make it difficult to lose weight and can contribute to weight gain.

Metabolic syndrome: Sleep apnea is often associated with metabolic syndrome, a cluster of conditions that includes high blood pressure, high blood sugar, and high cholesterol.

Other health problems: Sleep apnea can also be associated with a number of other health problems, including depression, anxiety, and cognitive impairment.

Sleep apnea is a major burden on the healthcare system. In the United States, it is estimated that sleep apnea costs the economy over $150 billion per year in healthcare costs and lost productivity.

The following are common symptoms of sleep apnea: snoring; daytime sleepiness; fatigue; morning headaches; difficulty concentrating; and waking up gasping for air.

There are a number of treatments for sleep apnea, including continuous positive airway pressure (CPAP) therapy, oral appliances including tongue retaining devices and surgery. However, many people with sleep apnea are undiagnosed or untreated.

There is evidence pointing to tongue muscle relaxation and sliding toward the back of throat causing obstructive sleep apnea. This is more pronounced in overweight individuals whose throat is excessively infiltrated with adipose tissue narrowing the airway and exaggerating the problem.

Effective, non-invasive and non-cumbersome, solutions which alleviate obstructive sleep apnea by preventing the tongue of a person from sliding toward the back of the throat can contribute to lessening the healthcare burden of sleep apnea.

### SUMMARY OF INVENTION

Therefore, the present invention provides a relatively simple approach to hold the tongue in place or pull it forward hence preventing its backward slide during sleep.

According to an aspect of the present invention, there is provided a tongue retaining device configured for reducing obstructive sleep apnea, comprising: a tongue piercing device; and one or more threads attached to the tongue piercing device and anchoring it to an anterior anchor point.

According to another aspect of the present invention, there is provided a tongue retaining device configured for reducing obstructive sleep apnea, comprising: a magnetic tongue piercing device; and one or more magnets are positioned anteriorly to attract the tongue piercing device and therefore preventing tongue contribution to obstructive sleep apnea.

According to yet another aspect of the present invention, there is provided a tongue retaining device configured for reducing obstructive sleep apnea, comprising: a tongue holding device, a pulling mechanism positioned anteriorly capable of pulling the tongue in anterior direction or stabilizing the position of the tongue to prevent tongue from contributing to obstructive sleep apnea.

### BRIEF DESCRIPTION OF THE DRAWINGS

FIG. 1 illustrates a barbell-style tongue ring device according to an embodiment of the present invention.

FIG. 2 illustrates a tongue adhesive pad according to an alternate embodiment of the present invention.

FIG. 3 illustrates a device with a mouthpiece according to another alternate embodiment of the present invention.

### DETAILED DESCRIPTION

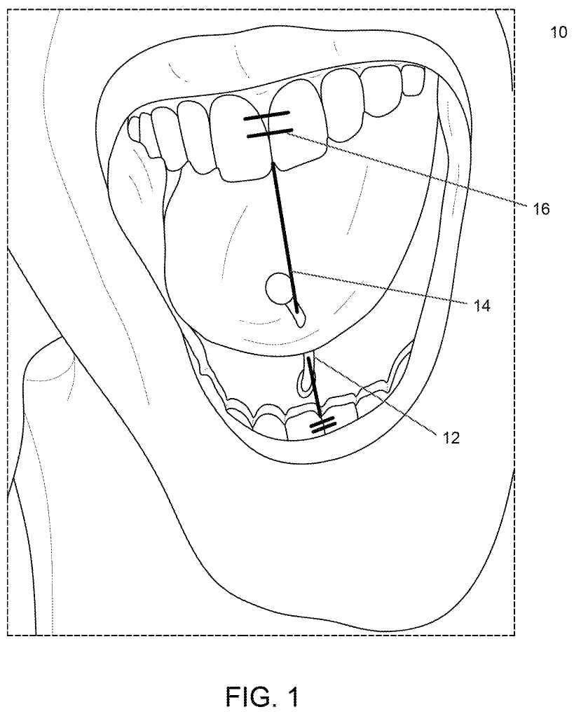

In FIG. 1, barbell-style tongue ring 12 is inserted into the pierced tongue of person 10. The barbell spans a piercing of the full thickness of the tongue. On the top and bottom of the tongue, elastic threads 14 are tied to the barbell. The elastic threads are then anchored to front teeth 16.

The tongue ring does not need to be permanent. The patient can use it only at night.

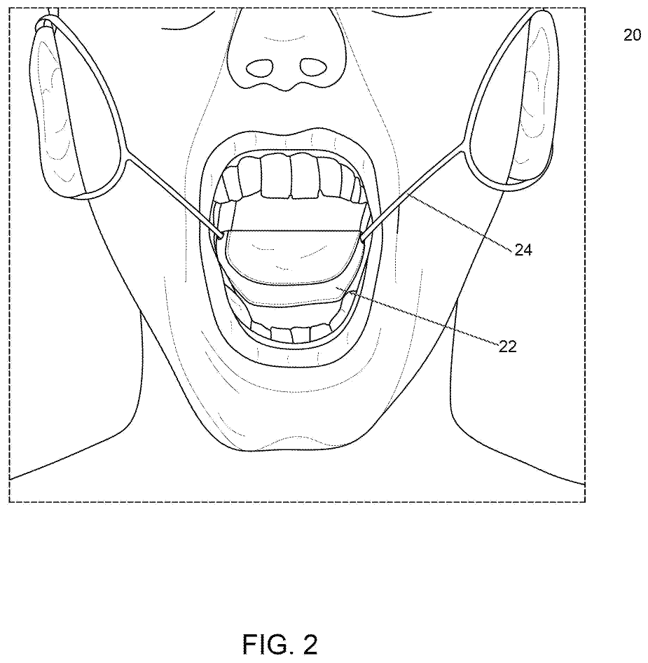

In FIG. 2 another embodiment is illustrated in which tongue sleeve or a toque adhesive pad 22 is used to anchor and pull the tongue of person 20 without a tongue ring. The pad or sleeve can be anchored to head-face mask 24 or a chocker necklace.

The tongue cap or adhesive may also have a strong skin adhesive on the side outside of the mouth to secure it with or without another securing mechanism.

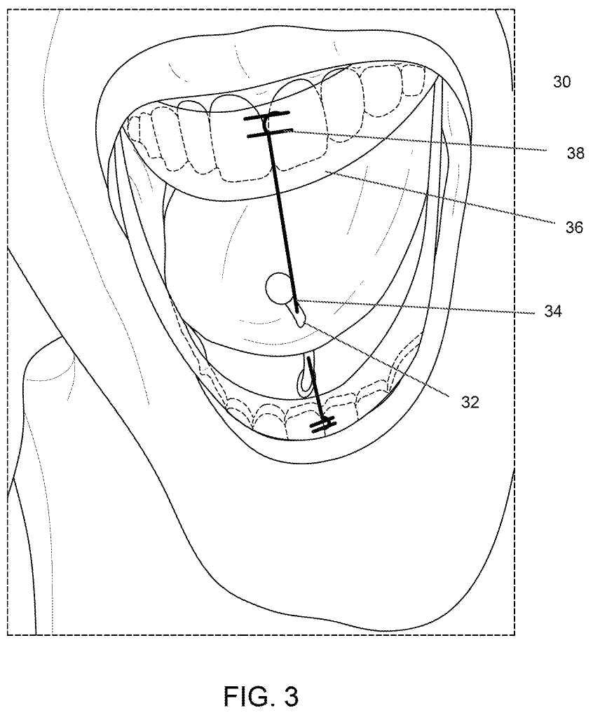

In FIG. 3, an embodiment is illustrated where instead of directly using the front teeth as an anchor mouthpiece 38 is inserted into the mouth of person 30 and anchor 38 is connected to elastic band 34 attached to barbell-style tongue ring 32.

Several more embodiments of this invention are disclosed.

In one alternate embodiment, a tongue ring is attached to the tongue with elastic threads which are anchored to the front teeth, and the threads are anchored to any other holding instruments like a neckband or a faceband (wrapping around the head at the mouth level to hold the threads).

In another alternate embodiment, the patient can either slide a captive bead ring with the bead popped out over the tip of the tongue, or buy a special suction ball to place on the center-top of the tongue so it looks like the tongue is pierced when it is not actually.

Another alternate embodiment is a hook type tongue ring that a patient slides in a tongue piercing and it is positioned in such a way that it does not require threads on both sides. On one side the hook anchors the tongue and the other side is attached to the thread that is anchored to the front teeth or other anchoring devices.

In another embodiment there is a single tread hook with the hook only on the upper side of the tongue.

In another embodiment the tongue ring is connected to the lower front teeth from the bottom side of the tongue.

In another embodiment the ring is a complete circular ring.

In another embodiment a ring is inserted under the tongue. It cannot be seen from the top of the tongue. The anchoring mechanism will be anchored to the lower front teeth

In another embodiment instead of the natural space between the front lower or upper teeth, an anchor mechanism is installed on any tooth inside the mouth (like a tooth cap or cover with a hook). This embodiment will be anchored by the hook and elastic band attached to the tongue ring.

In another embodiment instead of the front teeth the chin part of a head cap will be the anchor for the elastic band that is connected to the ring. The head cap can be one piece that one would wear before going to sleep. It can be like a thin breathable cotton facemask that is stretched over the head and face and pulls/keeps the tongue ring by the anchor.

In another embodiment the head cap can use one or multiple pieces attached with Velcro.

In another embodiment instead of the adhesive pad or the ring mechanisms the invention uses a simple wide plastic with a cut in the middle front of the band that a person can stick and squeeze their tongue in the hole. The elastic tightly holds the tongue and is secured by the rest of the band that goes around the neck. It is stretched tight or can be tightened on each side.

Another embodiment utilizes an alligator type clip in a person's mouth that can pull the tongue and is anchored to the a chocker necklace or a headband around the neck or head.

In another embodiment the entire tongue ring is removed and only a tongue threading device passed a thread through the tong piercing and anchors both sides to the front teeth. In this case the threads are soft elastic not causing pain on the tongue.

Yet another embodiment uses a strong magnet around the mouth positioned in such a way that it pulls the metal tongue ring towards the front teeth functioning as invisible threads.

Yet in another embodiment the tongue ring or piercing device itself is pulled out to be anchored to the front of the mouth which will result in a portion of tongue to stick out of mouth.

Yet in another embodiment the tongue ring has one or two metal or plastic bodies that clip to the front teeth. In this case swallowing may be more difficult as elasticity becomes an issue unless the connecting part to the clip can be stretched.

These inventions are superior to the tongue retaining devices in the market such as suction cups (pacifier like TRDs), which do not last on the tongue all night. Almost always they fall off in the middle of nice because of air or saliva leaking into the suction cup. It also keeps the mouth somewhat open that results in dry moth and irritation overnight.

The embodiments described above are given merely for example and for the purpose of facilitating the understanding of the present invention and are not intended to limit the interpretation of the present invention. The respective elements and their arrangements, materials, conditions, shapes, sizes, or the like of the embodiment are not limited to the illustrated examples but may be appropriately changed. Further, the constituents described in the embodiment may be partially replaced or combined together.

What is claimed is:

1. A tongue retaining device configured for reducing obstructive sleep apnea, comprising:
a tongue piercing device; and
one or more threads attached to the tongue piercing device and anchoring it to an anterior anchor point.

2. The tongue retaining device of claim 1 wherein the tongue piercing device pierces the full thickness of the tongue.

3. The tongue retaining device of claim 1 wherein the tongue piercing device pierces the sublingual frenum of the tongue.

4. The tongue retaining device of claim 1 wherein the thread is attached to the both sides of the piercing device.

5. The tongue retaining device of claim 1 wherein the thread is attached to the one side of the piercing device.

6. The tongue retaining device of claim 1 wherein the piercing device has round ends to prevent injury.

7. The tongue retaining device of claim 1 wherein one or more of the threads attaches to a hook on the tongue piercing device.

8. The tongue retaining device of claim 1 wherein one or more of the threads loops through the tongue piercing device.

9. The tongue retaining device of claim 1 wherein the anterior anchoring point is one or more of front lower teeth, front upper teeth, a mouthpiece, a facemask outside of the mouth, a chocker necklace, or an adhesive surface placed on the skin outside of the mouth.

10. A tongue retaining device configured for reducing obstructive sleep apnea, comprising:
a magnetic tongue piercing device; and
one or more magnets are positioned anteriorly to attract the tongue piercing device and therefore preventing tongue contribution to obstructive sleep apnea.

11. The tongue retaining device of claim 1 wherein one of the magnets is a mouthpiece.

12. The tongue retaining device of claim 1 wherein one of the magnets is worn around the anterior part of the neck or submandibular area.

13. A tongue retaining device configured for reducing obstructive sleep apnea, comprising:
a tongue holding device,
a pulling mechanism positioned anteriorly capable of pulling the tongue in anterior direction or stabilizing the position of the tongue to prevent tongue from contributing to obstructive sleep apnea.

14. The tongue retaining device of claim 13 wherein the tongue holding device is a tongue sleeve.

15. The tongue retaining device of claim 13 wherein the tongue holding device is a tongue adhesive.

16. The tongue retaining device of claim 15 wherein the tongue holding device a tongue adhesive pad with additional properties comprising one or more of antibacterial effects, anti-odor effects, anti-dry mouth effects, and therapeutic effects.

17. The tongue retaining device of claim 13 wherein the tongue holding device is a clamp.

18. The tongue retaining device of claim 13 wherein the tongue holding device is an stretchable elastic band with a hole capable of receiving and wrapping around the tongue.

19. The tongue retaining device of claim 13 wherein the pulling or stabilizing mechanism is anchored to a facemask, a chocker necklace, a skin adhesive piece.

20. The tongue retaining device of claim 13 wherein the tongue holding device is a stretchable elastic band built in a mouthpiece.
